(19) United States
(12) Patent Application Publication
(10) Pub. No.: US 2014/0309257 A1
(43) Pub. Date: Oct. 16, 2014

(54) TOPOISOMERASE INHIBITORS AND METHODS OF MAKING AND USE AS THERAPEUTIC AGENTS

(71) Applicant: THE REGENTS OF THE UNIVERSITY OF COLORADO, a body corporate, Denver, CO (US)

(72) Inventor: Daniel V. Labarbera, Centennial, CO (US)

(21) Appl. No.: 14/357,249

(22) PCT Filed: Nov. 13, 2012
(86) PCT No.: PCT/US12/64869

§ 371 Date: May 9, 2014

Related U.S. Application Data
(60) Provisional application No. 61/558,439, filed on Nov. 11, 2011.

(51) Int. Cl. C07D 401/12; C07D 215/44; A61K 31/4706; A61N 5/10; C07D 215/48; A61K 31/47; C07D 215/38; A61K 45/06; A61K 31/475

(52) U.S. Cl. CPC C07D 401/12; A61K 45/06; C07D 215/44; A61K 31/4706; A61K 31/475; C07D 215/48; A61K 31/47; C07D 215/38; A61N 5/10
USPC 514/313; 546/160; 546/162; 546/170; 514/311; 546/171; 600/1

(57) ABSTRACT

The invention provides methods of inhibiting the growth or metastasis of cancer in a mammal by inhibiting TopoIIa in the mammal. The invention also provides small molecule inhibitors of TopoIIa useful in the methods of the invention and pharmaceutical compositions containing the therapeutically effective compounds and methods of using the same.

### TECHNICAL FIELD

The invention relates to therapeutic compounds, pharmaceutical compositions containing the same and their use in the prevention or treatment of cancer.

### BACKGROUND OF INVENTION

Type IIα DNA topoisomerase (TopoIIα) plays a critical role in cell cycle progression by relieving various types of topological strain induced in DNA double strands during replication, transcription, and chromosome segregation, making it a crucial target for chemotherapy in several cancers. The nuclear homodimer causes transitory double-stranded breaks in DNA, allowing it to pass a second double strand through before re-ligating the broken strand, fueled by ATP hydrolysis. This process generates an intermediate complex in which TopoIIα is covalently bound to the cleaved DNA, known as the cleaved complex. Conventional TopoIIα poisons, such as doxorubicin and etoposide, act by stabilizing this complex, leading to its accumulation, DNA lesions, and ultimately, apoptosis. Accumulation of this cleaved complex leads to chromosomal translocations and non-specific cytotoxicity, contributing to adverse side effects including secondary malignancies.

Multi-drug resistance (MDR) is a major problem associated with the existing clinically used TopoIIα inhibitors. The most common mode of clinical drug resistance is due to the ABCB1 gene product P-glycoprotein (Pgp) efflux pump. Another mode of MDR is associated with alterations in topoisomerase II, including TopoIIα phosphorylation (which results in increased catalytic activity) and TopoIIα complex formation with cellular proteins that alter its function without phosphorylation (which results in increased catalytic activity—shown to promote MDR in multiple types of cancer).

Thus, there remains a need for improved TopoIIα inhibiting compounds that can overcome drug resistance in cancer cells and can be successfully used to prevent or treat tumors and metastatic cancers.

### SUMMARY OF INVENTION

The present invention provides molecules that can inhibit TopoIIα, as well as therapeutic uses of these molecules to prevent or slow the growth and metastasis of cancer in a mammal.

Neoamphimedine (neo), a TopoIIα inhibitor from Xestospongia sp was first isolated and reported in 1999 as an antineoplastic agent.

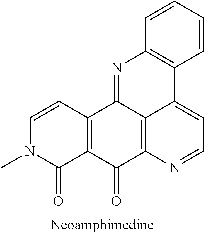 [SMILES: Cn1ccc2c(c1=O)C(=O)c1nccc3c1c-2nc1ccccc13]

Neo has been shown to be as effective as etoposide at inhibiting the growth of xenograft tumors in mice. Neo acts to inhibit TopoIIα without stabilizing the cleaved complex, or causing significant DNA strand breaks or intercalation of DNA below 100 μM concentrations. Neo is not a substrate for Pgp-dependent MDR, and it has been shown to be as efficacious as clinically used TopoIIα poisons both in vitro and in vivo (Marshall K M, Matumoto S S, Holden J A, Concepcion G P, Tasdemir D, et al. (2003), The anti-neoplastic and novel topoisomerase II-mediated cytotoxicity of neoamphimedine, a marine pyridoacridine. Biochem Pharmacol 66: 447-458). The present inventor has shown that neo inhibits TopoIIα ATPase function with an IC₅₀ of 2 μM and a corresponding Kᵢ of 0.8±0.3 μM. The inventor has shown that the primary anti-tumor activity of neo is the ATP-competitive inhibition of TopoIIα function. This inhibition of the TopoIIα ATPase function is distinct from the inhibition of the topological activity, which is the mechanism of most of the currently-known and used TopoIIα inhibitor anti-cancer compounds, which leads to drug resistance, adverse drug effects and a lack of effectiveness in the treatment of metastatic cancers.

The present inventors have synthesized neo (LaBarbera, D. V.; Bugni, T. S.; Ireland, C. M., The total synthesis of neoamphimedine. J Org Chem 2007, 72, 8501-8505), designed and synthesized neo derivatives, and conducted in vitro efficacy testing, including computational docking using the ATPase sites of TopoIIα (FIG. 1). Rational drug design resulted in two derivatives, AA-26 and AA-67. These derivatives show improved binding affinities and increased active site interactions compared with neo. Further rational design based on the pharmacophore of neo allowed the elimination of the iminoquinone moiety from the molecule. Quinones and iminoquinones have been shown to produce adverse toxicity due to reactive oxygen species. Additionally, the X and R groups can be easily modified to optimize active site binding. Derivatives AA-26 and AA-67 were synthesized using synthetic methodology developed to prepare neo (LaBarbera D V, Bugni T S, Ireland C M (2007), The Total Synthesis of Neoamphimedine. J Org Chem 72: 8501-8505), and the key neo intermediate shown in FIG. 2.

These compounds have been evaluated in vitro for their efficacy in inhibiting TopoIIα dependent DNA decatenation, using structure based drug design coupled with in vitro, 3D-cell based animal models, including: pharmacokinetics, and tumor and metastasis models using primary tumors from humans explanted into nude mice. These efficacy studies lead to the therapeutic methods of the present invention.

Thus, the present invention provides compounds that inhibit TopoIIα, and target both localized tumors and invasive metastatic tumors, and pharmaceutically acceptable salts thereof.

The invention also provides pharmaceutical compositions containing these compounds, and methods of using these compounds and pharmaceutical compositions to treat cancer and prevent cancer metastasis and tumor proliferation. These compounds have a lower incidence of adverse side effects than the currently used TopoII inhibitors, etoposide and doxorubicin.

One embodiment of the invention is a method of treating a cancer by administering to a mammal in need of such treatment, an effective amount of a compound that inhibits TopoIIα enzymatic activity. In one aspect of this embodiment, the compound inhibits at least TopoIIα ATPase activity, thereby inhibiting the growth or metastasis of a cancer. In a specific aspect of this embodiment, the compound inhibits the ATPase activity of TopoIIα. In one embodiment, the cancer is a human colon cancer. In another embodiment, the cancer is a human breast cancer. In another embodiment, the cancer is a secondary cancer metastasized from a primary cancer.

Compounds of the invention that inhibit TopoIIα activity include compounds having the chemical structure of Formula I:

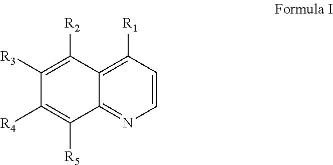

and pharmaceutically acceptable salts, solvates, tautomers, racemates, pure enantiomers, diastereoisomers or N-oxides thereof,

wherein:

R₁ is: H, —NO₂, —NHC(O)R₁₀, —NH₂, —NHR₈, or —NR₈R₉;

or R₁ is: phenyl or substituted phenyl, wherein the substituents are independently selected from amides, —NCOCH₃, —NCOR₁₃, —NCOR₁₃, and —C₁₋₆ alkyl;

R₂ is: H, —OR₁₄ or —NR₁₄, wherein R₁₄ is halogen, C₁₋₆ alkyl, C₃₋₆ cycloalkyl, cyano, or optionally substituted phenyl, wherein the optional substituents are independently selected from halogens, hydroxyl, C₁₋₆ alkoxy;

R₃ is: H, halogen, C₁₋₆ alkyl, C₃₋₆ cycloalkyl, or cyano;

R₄ is: H, sulfonamide, phosphamide, sulfonate ester, phosphonate ester, P-amido-imido-diphosphoric acid, imido-disulfamide, —NHR₈, —NHR₈R₉; —NH—SO₂—R₁₁, NH—PO₂—R₁₁, —COR₁₂, or —NHCOR₁₂;

or R₃ and R₄ together form a C₅₋₁₀ membered cycloalkyl, aryl or heteroaryl ring with one or more optional heteroatoms independently selected from nitrogen, oxygen and sulfur, and the optional substituents are independently selected from halogens, hydroxyl, C₁₋₃ alkoxy, cyano, C₁₋₆ alkyl or C₃₋₆ cycloalkyl, and aryl;

R₅ is: H, —OR₁₅ or —NR₁₅, wherein R₁₅ is halogen, C₁₋₆ alkyl, C₃₋₆ cycloalkyl, or cyano;

R₈ and R₉ are independently selected from H, or optionally substituted C₁₋₆ alkyl, C₄₋₁₀ aryl, phenyl or indole, wherein the optional substituents are independently selected from halogens, hydroxy, C₁₋₃ alkoxy, phenyl or substituted phenyl, wherein the substituents are individually selected from amides, —NCOCH₃, —NCOR₁₃, and —C₁₋₆ alkyl;

R₁₀ is: H or optionally substituted C₁₋₆, phenyl, benzyl, and naphthyl, imidazole, pyridyl, pyrimidine, pyrolidine, quinoxaline, and quinoline ring systems wherein the optional substituents are independently selected from amino, halogens, hydroxyl, C₁₋₆ alkoxy;

R₁₁ and R₁₂ are independently selected from H, hydroxyl, optionally substituted C₁₋₆ alkyl, phenyl, benzyl, and naphthyl, imidazole, pyridyl, pyrimidine, pyrolidine, quinoxaline, and quinoline ring systems wherein the optional substituents are independently selected from amino, halogens, hydroxyl, and C₁₋₆ alkoxy;

R₁₃ is: C₁₋₆ alkyl, C₄₋₆ aryl.

In a specific embodiment of the invention, the compound is a compound of Formula I, wherein R₁ is —N—C(O)CH₃, R₂ is —CH₃, R₃ is —H, R₄ is —COOH, —C(O)NH₂, —NHC(O)CH₃ and R₅ is —OH, or a pharmaceutically acceptable salt thereof. In another specific embodiment of the invention, the compound is a compound of Formula I, wherein R₁ is —CH₃, R₂ is —CH₃, R₃ is —H, R₄ is —NHCOCH₃, and R₅ is —OH, or a pharmaceutically acceptable salt thereof.

For example, another specific embodiment of the invention is a compound of Formula I, having the structure:

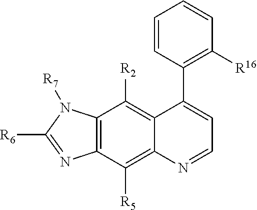

or pharmaceutically acceptable salts, solvates, tautomers, racemates, pure enantiomers, diastereoisomers or N-oxides thereof,

wherein:

R₁₆ is: amide, —NCOCH₃, —NCOR₁₃, —NCOR₁₃, and —C₁₋₆ alkyl;

R₂ is: H, —OR₁₄ or —NR₁₄, wherein R₁₄ is halogen, C₁₋₆ alkyl, C₃₋₆ cycloalkyl, cyano, or optionally substituted phenyl, wherein the optional substituents are independently selected from halogens, hydroxyl, C₁₋₆ alkoxy;

R₅ is: H, —OR₁₅ or —NR₁₅, wherein R₁₅ is halogen, C₁₋₆ alkyl, C₃₋₆ cycloalkyl, or cyano;

R₆ is: sulfonamide, phosphamide, sulfonate ester, phosphonate ester, P-amidoimidodiphosphoric acid, imidodisulfamide, —N—SO₂—R₁₁, —N—PO₂—R₁₁—C(O)R₁₁, or —NHC(O)—R₁₂;

or R6 is C₁₋₆ optionally substituted with sulfonamide, phosphamide, sulfonate ester, phosphonate ester, P-amidoimidodiphosphoric acid, imidodisulfamide, —N—SO₂—R₁₁, —N—PO₂—R₁₁—C(O)R₁₁, or —NHC(O)—R₁₂;

R₇ is: C₁₋₆ alkyl (wherein the optional substituent(s) is/are selected from halogens, hydroxy, C₁₋₃ alkoxy.

Other exemplary compounds of the invention include compounds having the structures shown in FIG. 5, and pharmaceutically acceptable salts, solvates, tautomers, racemates, pure enantiomers, diastereoisomers or N-oxides thereof.

The invention also provides pharmaceutical compositions containing one or more of the compounds of the invention with at least one pharmaceutically-acceptable carrier. Thus, in one aspect of the invention, at least one compound of the invention is administered to a mammal in a pharmaceutical composition of the invention.

Another embodiment of the invention is a method of preventing or treating metastatic cancers, by administering a therapeutically effective amount of one compound of the invention, or a pharmaceutically acceptable salt thereof, to a mammal in need of such treatment, or suspected of having a primary cancer or a metastasis of a primary cancer.

Another embodiment of this invention is a method of treating cancer in a mammal by administering a therapeutically effective combination of at least one of the compounds of the invention and one or more known anti-cancer treatments. For example, other anti-cancer treatments may include doxorubicin, etoposide, ICRF-193, bevacizumab, carboplatin, cisplatin, ifosfamide, paclitaxel, pazopanib, vinblastine or vincristine, surgery, chemotherapy, radiation, immunotherapy, or combinations thereof.

Also provided herein are methods for the prevention, treatment or prophylaxis of cancer in a mammal, comprising administering to the mammal in need thereof, therapeutically-effective amounts of any of the pharmaceutical compositions of the invention.

Also provided herein are methods for preventing the metastasis of a cancer in a mammal by administering to the mammal therapeutically-effective amounts of at least one compound of the invention, including, for example, the pharmaceutical compositions containing at least one compound of the invention. Similarly, provided herein are methods for treating a metastatic cancer in a mammal by administering to the mammal therapeutically-effective amounts of at least one compound of the invention, including, for example, the pharmaceutical compositions containing at least one compound of the invention.

In these embodiments of the invention, the cancer may be a cancer selected from breast, colon, epidermoid, prostate, pancreatic, leukemia, ovarian, small cell lung, cervical, neuroblastoma, endometrial, melanoma, renal and peritoneal cancers.

Also provided herein are packages containing a pharmaceutical composition comprising therapeutically-effective amounts of at least one compound of the invention, optionally together with at least one pharmaceutically acceptable carrier. The pharmaceutical compositions may be administered separately, simultaneously or sequentially, with other compounds or therapies used in the prevention, treatment or amelioration of cancer. These packages may also include prescribing information and/or a container. If present, the prescribing information may describe the administration, and/or use of these pharmaceutical compositions alone or in combination with other therapies used in the prevention, treatment or amelioration of cancer.

Other aspects of the invention will be set forth in the accompanying description of embodiments, which follows and will be apparent from the description or may be learnt by the practice of the invention. However, it should be understood that the following description of embodiments is given by way of illustration only since various changes and modifications within the spirit and scope of the invention will become apparent to those skilled in the art and are encompassed within the scope of this invention.

### BRIEF DESCRIPTION OF DRAWINGS

FIG. 1 shows the crystal structure (PDB: 1ZXM) of the N-terminal ATPase sites of TopoIIα bound to neo (circled) non-solvent binding energy is −66 kcal/mol.

FIG. 2 shows the pharmacophore of neo and retro synthetic methodology to prepare neo and derivatives from the intermediate shown in the inset.

FIG. 3 shows the synthetic route of neo derivative AA67.

FIG. 4 shows the synthetic route of neo derivative AA26.

FIG. 5 shows 12 compounds of the invention synthesized and tested in vitro.

FIG. 6 shows a synthesis scheme for compounds of the invention.

FIG. 7 shows the results of in vitro testing of compounds of the invention.

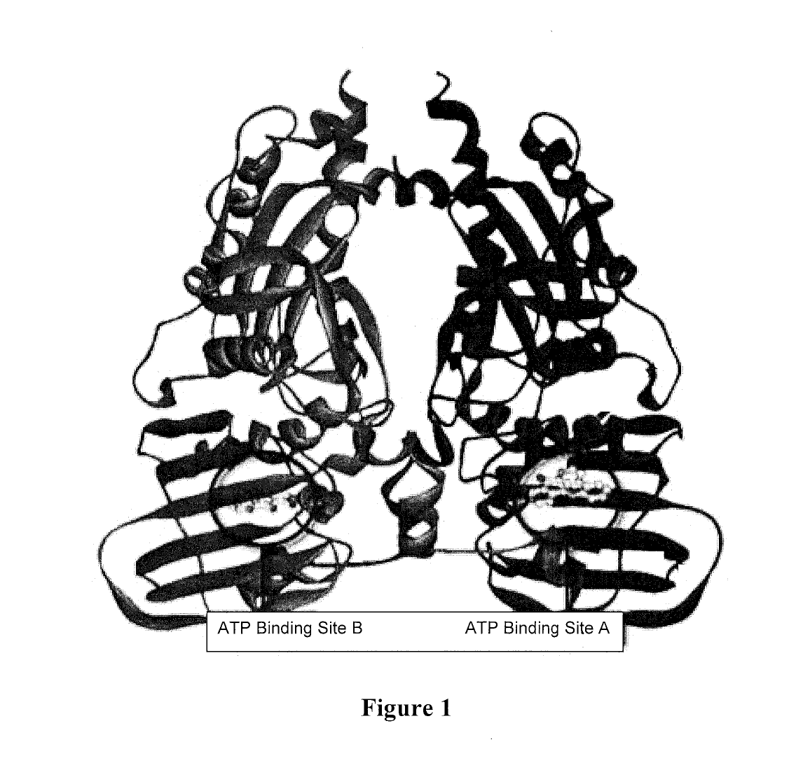

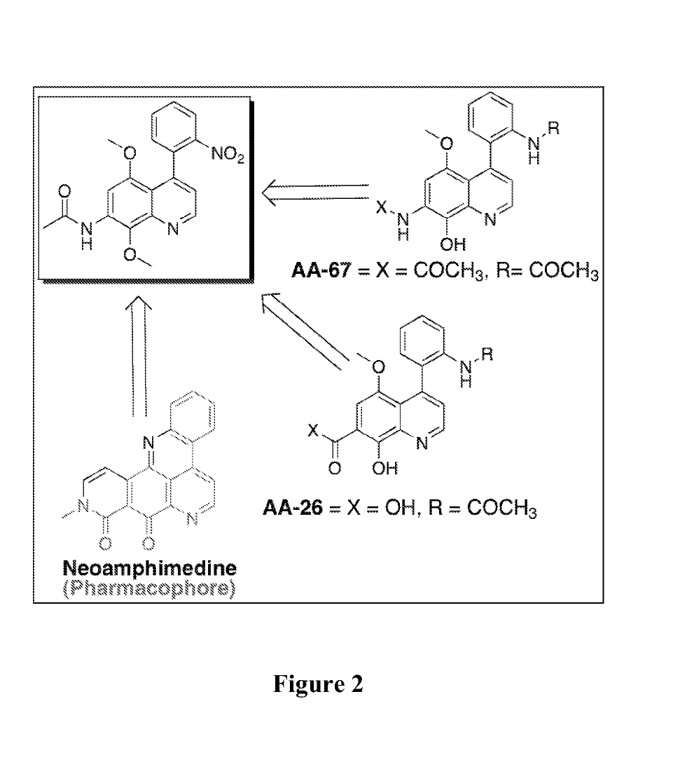

### DESCRIPTION OF EMBODIMENTS

One of the exclusive functions of TopoIIα is to decatenate (i.e., disentangle) sister chromatids before segregation. Metnase, a double strand break repair factor, interacts with TopoIIα and promotes TopoIIα-dependent DNA decatenation, and recent studies of this interaction show that Metnase can promote tumor cell growth in the presence of conventional TopoIIα poisons, which may be an important factor in multidrug resistance (MDR) in proliferating tumors overexpressing Metnase. Thus, without intending to be bound by any theory, the inventors hypothesized that protein-protein interactions with TopoIIα, with the exception of the N-terminal ATPase binding sites required for functional protein interactions, convey the MDR cancer cell phenotype.

Additionally, it is known that prior to metastasis, primary tumors undergo epithelial-mesenchymal transition (EMT) acquiring the mesenchymal phenotype. Inherent to the mesenchymal phenotype is an increased invasive potential and multi-drug resistance (MDR) to apoptosis and conventional chemotherapy. The present inventor has discovered and identified topoisomerase IIα (TopoIIα) as a key regulator of mesenchymal homeostasis in metastatic cancer. This is supported by evidence suggesting that TopoIIα changes roles from a master regulator of cell proliferation in localized cancer to a master regulator of mesenchymal gene expression in primary and metastatic cancer. Despite the importance of TopoIIα in mesenchymal gene regulation, current TopoIIα drugs have been relatively ineffective at treating metastatic cancer due to loss or limited efficacy of these drugs in treating cancers displaying the MDR phenotype.

Therefore, in order to develop systemic therapies that can suppress or revert the metastatic phenotype in the treatment of cancers that display or have developed the MDR phenotype, the inventors have designed anti-cancer TopoIIα inhibitors that bind to and inhibit TopoIIα at the N-terminal ATPase binding site, thereby avoiding the loss of efficacy often seen with, for example, etoposide, doxorubicin and ICRF-193.

The present inventors have identified neoamphimedine (neo), a novel marine alkaloid, as a compound that significantly inhibits the invasive potential of triple negative MDA-MB-231 breast carcinoma spheroids through reversion of EMT, and have demonstrated that neoamphimedine (neo) increases the apparent Kₘ of TopoIIα for ATP by approximately 2-fold over a concentration range from 0-10 μM, indicative of an ATP-competitive mode of inhibition. Computational docking studies identified the ATPase sites as the binding sites of neo, including a network of hydrogen bonds with Ser148, Ser149, and Asn150, Asn91, and pi-cation interactions with Mg²⁺.

Neo has previously been shown to be as efficacious as etoposide and 9-aminocamptothecin in human epidermoid-nasopharyngeal tumor (KB) and human colon tumor (HCT-116) xenografts, respectively (Marshall K M, Matumoto S S, Holden J A, Concepcion G P, Tasdemir D, et al. (2003), The anti-neoplastic and novel topoisomerase II-mediated cytotoxicity of neoamphimedine, a marine pyridoacridine. Biochem Pharmacol 66: 447-458). Despite these impressive in vivo results, further development of neo as an anti-tumor agent were limited for a number of reasons, including limited supply of the natural product, incomplete understanding of the mechanism of action, and unknown anti-metastatic activity.

The present inventors have used rational drug design to produce neo derivatives that showed improved binding affinities and increased active site interactions as compared to neo. These derivatives were synthesized from a key neo intermediate and were shown to be significantly more potent at inhibiting TopoIIα dependent DNA decatenation than neo.

Thus, the present invention provides methods of treating cancer in an individual by administering an effective amount of a compound of the invention to an individual in need thereof. In these methods, the compounds may be administered as or within a pharmaceutical composition. The present invention also provides methods of inhibiting the growth and/or metastasis of a cancer by administering an effective amount of a compound or composition of the invention to an individual in need thereof. These compounds preferably inhibit TopoIIα in a mammal. These methods, as well as compounds and compositions useful in these methods, may be used to treat mammals diagnosed with, or suspected of having, a cancer. In a specific aspect, the invention is a method of inhibiting the growth and/or metastasis of a cancer by administering at least one compound of the invention, or pharmaceutically-acceptable salts thereof to an individual in need thereof.

In a specific embodiment, the compounds of the present invention are competitive inhibitors of ATP for the N-terminal ATPase binding site of TopoIIα. These compounds are significantly more potent than neo and overcome drug resistance in cancer cells. Therefore, these compounds are potent anti-cancer compounds that can be successfully used to prevent or treat tumors, particularly metastatic cancers, including instances in which the cancer displays resistance to multiple anti-cancer drugs (MDR).

As used herein, the term “compound” means a chemical or biological molecule such as a simple or complex organic molecule, a peptide, a protein or an oligonucleotide.

The term “alkyl” as used herein is directed to a saturated hydrocarbon group (designated by the formula CₙH₂ₙ₊₁) which is straight-chained, branched or cyclized (“cycloalkyl”) and which is unsubstituted or substituted, i.e., has had one or more of its hydrogens replaced by another atom or molecule.

“Aryl” designates either the 6-carbon benzene ring or the condensed 6-carbon rings of other aromatic derivatives (see, e.g., Hawley's Condensed Chemical Dictionary (13 ed.), R. J. Lewis, ed., J. Wiley & Sons, Inc., New York (1997)). Aryl groups include, without limitation, phenyl, naphthyl, indanyl and indenyl. “Substituted aryl” means that one or more hydrogen atoms on the designated aryl substituent is replaced with a selection from the indicated group, provided that the designated atom's normal valency is not exceeded, and that the substitution results in a stable compound. Exemplary aryl substituents include, but are not limited to, hydroxy, mercapto, amino and substituted amino, nitro, carboxylic acid, amide or ester derivatives, sulfonic acid, halide, trihalomethyl, C₁₋₆ alkyl, C₁₋₆ alkenyl, C₁₋₄ alkoxy, and C₃₋₈ cycloalkyl.

The term “heteroaryl” refers to monocyclic or polycyclic groups having at least one aromatic ring structure and including one or more heteroatoms and preferably one to fourteen carbon atoms. Illustrative examples of heteroaryl groups include, but are not limited to, furanyl, imidazolyl, indanyl, indolyl, indazolyl, isoxazolyl, isoquinolyl, oxazolyl, oxadiazolyl, pyrazinyl, pyridyl, pyrimidinyl, pyrrolyl, pyrazolyl, quinolyl, quinoxalyl, tetrazolyl, thiazolyl, thienyl, and the like.

The term “heterocycle” or “heterocyclic” or “heterocyclic moiety” refers to ring-containing monovalent and divalent radicals having one or more heteroatoms, independently selected from N, O and S, as part of the ring structure and comprising at least 3 and up to about 20 atoms in the rings preferably 5- and 6-membered rings. Heterocyclic moieties may be saturated or unsaturated, containing one or more double bonds, and heterocyclic moieties may contain more than one ring. Heterocyclic moieties include for example monocyclic moieties such as: aziridine, oxirane, thiirane, azetidine, oxetane, thietane, pyrrolidine, pyrroline, imidazolidine, pyrazolidine, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide. In addition heterocyclic moieties include heteroaryl rings such as: pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl. Additionally, heterocyclic moieties encompass polycyclic moieties such as: indole, indoline, quinoline, tetrahydroquinoline, isoquinoline, tetrahydroisoquinoline, 1,4-benzodioxan, coumarin, dihydrocoumarin, benzofuran, 2,3-dihydrobenzofuran, 1,2-benzisoxazole, benzothiophene, benzoxazole, benzothiazole, benzimidazole, benzotriazole, thioxanthine, carbazole, carboline, acridine, pyrolizidine, and quinolizidine.

“Alkenyl” as used herein by itself or as part of another group refers to straight or branched chain substituent of 2 to 12 carbons, preferably 2 to 5 carbons, in the normal chain, which include one to six double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, and the like, which may be substituted in the same manner as that described for alkyl groups.

The term “cycloalkyl” as employed herein alone or as part of another group includes saturated cyclic hydrocarbon groups or partially unsaturated (containing 1 or 2 double bonds) cyclic hydrocarbon groups, containing one ring and a total of 3 to 7 carbons, preferably 3 to 6 carbons, forming the ring, which includes cyclopropyl, cyclobutyl, cyclopentyl, cyclohexyl, cycloheptyl, cyclopentenyl and cyclohexenyl, which may be substituted in the same manner as that described for alkyl groups.

“Cycloalkenyl” means C₃₋₈ cycloalkyl containing one or more double bonds.

“Alkoxy” means —OR where R is alkyl as defined above, e.g., methoxy, ethoxy, propoxy, 2-propoxy, acetyl and the like.

“Alkynyl” means a linear monovalent hydrocarbon of two to six carbon atoms or a branched divalent hydrocarbon of three to six carbon atoms, containing at least one triple bond, e.g., ethynyl, propynyl, and the like.

The term “halogen” refers to nonmetal elements from Group 17 of the periodic table, including fluorine, F; chlorine, Cl; bromine, Br; iodine, I; and astatine, At.

The term “amino acid side chain” refers to the side chain of any of the known alpha-amino acids such as the side chain of arginine, histidine, alanine, glycine, lysine, glutamine, leucine, valine, serine, homoserine, allothreonine, naphthylalanine, isoleucine, phenylalanine and the like. In instances in which a compound is synthesized or derivatized to include an amino acid side chain, the side chain used is preferably chosen from the side chains of the naturally-occurring amino acids.

Substituent groupings, e.g., C₁₋₄ alkyl, are known, and are hereby stated, to include each of their individual substituent members, e.g., C₁ alkyl, C₂ alkyl, C₃ alkyl and C₄ alkyl.

“Substituted” means that one or more hydrogen atoms on the designated atom is replaced with a selection from the indicated group, provided that the designated atom's normal valency is not exceeded, and that the substitution results in a stable compound.

“Unsubstituted” atoms bear all of the hydrogen atoms dictated by their valency. When a substituent is keto, then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Pharmaceutically-acceptable salts” refer to derivatives of the disclosed compounds wherein the parent compound is modified by making acid or base salts thereof Examples of pharmaceutically acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of compounds, suitable for use in contact with the tissues of human beings and animals without excessive toxicity, irritation, allergic response, or other problem or complication, commensurate with a reasonable benefit/risk ratio.

Pharmaceutically-acceptable salt forms of compounds provided herein are synthesized from the compounds of the invention which contain a basic or acidic moiety by conventional chemical methods. Generally, such salts are, for example, prepared by reacting the free acid or base forms of these compounds with a stoichiometric amount of the appropriate base or acid in water or in an organic solvent, or in a mixture of the two;

generally, nonaqueous media like ether, ethyl acetate, ethanol, isopropanol, or acetonitrile are preferred. Lists of suitable salts are found in at page 1418 of Remington's Pharmaceutical Sciences, 17th ed., Mack Publishing Company, Easton, Pa., 1985.

The term “therapeutically-effective amount” or “effective amount” of a compound of this invention means an amount effective to inhibit the formation or progression of cancer following administration to a mammal having a cancer.

By “side chain” of an amino acid is meant that portion of the amino acid attached to the common NH₂—CH—COOH backbone of all of the amino acids. For instance, the side chain of glycine is —H, the side chain of alanine is —CH₃, and the side chain of serine is —CH₂OH. Exemplary side chains of amino acids useful in the compounds of the invention are side chains of glycine, alanine, valine, norvaline, α-aminoisobutyric acid, 2,4-diaminobutyric acid, 2,3-diaminobutyric acid, leucine, isoleucine, norleucine, serine, homoserine, threonine, aspartic acid, asparagine, glutamic acid, glutamine, lysine, hydroxylysine, histidine, arginine, homoarginine, citrulline, phenylalanine, p-aminophenylalanine, tyrosine, tryptophan, thyroxine, cysteine, homocysteine, methionine, penicillamine or ornithine.

It will be appreciated by those skilled in the art that compounds of the invention having a chiral center may exist in, and may be isolated in, optically active and racemic forms. It is to be understood that the compounds of the present invention encompasses any racemic, optically-active, regioisomeric or stereoisomeric form, or mixtures thereof, which possess the therapeutically useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase). It is also to be understood that the scope of this invention encompasses not only the various isomers, which may exist but also the various mixtures of isomers, which may be formed. For example, if the compound of the present invention contains one or more chiral centers, the compound can be synthesized enantioselectively or a mixture of enantiomers and/or diastereomers can be prepared and separated. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the compounds is based on the differences in the physical properties of diastereomers by attachment, either chemically or enzymatically, of an enantiomerically pure moiety resulting in forms that are separable by fractional crystallization, distillation or chromatography.

The compounds used in making the pharmaceutical compositions of the present invention may synthesized as described in the Examples section of this description from commercially available materials in ways well known to those skilled in the art of organic synthesis. The compounds of the invention may be prepared using the reactions performed in solvents appropriate to the reagents and materials employed and suitable for the transformation being effected. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

Also provided herein are pharmaceutical compositions containing compounds of the invention and at least one pharmaceutically-acceptable carrier, which are media generally accepted in the art for the delivery of biologically active agents to animals, in particular, mammals. Pharmaceutically-acceptable carriers are formulated according to a number of factors well within the purview of those of ordinary skill in the art to determine and accommodate. These include, without limitation: the type and nature of the active agent being formulated; the subject to which the agent-containing composition is to be administered; the intended route of administration of the composition; and, the therapeutic indication being targeted. Pharmaceutically-acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically-acceptable carriers, and factors involved in their selection, are found in a variety of readily available sources, such as Remington's Pharmaceutical Sciences, 17th ed., Mack Publishing Company, Easton, Pa., 1985.

This invention further provides a method of treating a mammal afflicted with cancer or preventing the metastasis of such cancer in a mammal, which includes administering to the mammal a pharmaceutical composition provided herein. Such compositions generally comprise a therapeutically effective amount of a compound of the invention in an amount effective to prevent, ameliorate, lessen or inhibit the cancer. Such amounts typically comprise from about 0.1 to about 100 mg of the compound per kilogram of body weight of the mammal to which the composition is administered. Therapeutically-effective amounts can be administered according to any dosing regimen satisfactory to those of ordinary skill in the art.

Administration may be, for example, by various parenteral means. Pharmaceutical compositions suitable for parenteral administration include various aqueous media such as aqueous dextrose and saline solutions; glycol solutions are also useful carriers, and preferably contain a water soluble salt of the active ingredient, suitable stabilizing agents, and if necessary, buffering agents. Antioxidizing agents, such as sodium bisulfite, sodium sulfite, or ascorbic acid, either alone or in combination, are suitable stabilizing agents; also used are citric acid and its salts, and EDTA. In addition, parenteral solutions can contain preservatives such as benzalkonium chloride, methyl- or propyl-paraben, and chlorobutanol.

Alternatively, compositions can be administered orally in solid dosage forms, such as capsules, tablets and powders; or in liquid forms such as elixirs, syrups, and/or suspensions. Gelatin capsules can be used to contain the active ingredient and a suitable carrier such as, but not limited to, lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

A preferred formulation of the invention is a mono-phasic pharmaceutical composition suitable for parenteral or oral administration for the prevention, treatment or prophylaxis of a cancer, consisting essentially of a therapeutically-effective amount of a compound of the invention, and a pharmaceutically acceptable carrier.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn may depend upon crystal size and crystalline form. Alternatively, delayed absorption of a parenterally-administered drug is accomplished by dissolving or suspending the drug in an oil vehicle.

Injectable depot forms are made by forming microencapsulated matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the therapeutic compounds of the present invention.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations include those suitable for administration by inhalation or insufflation or for nasal or intraocular administration. For administration to the upper (nasal) or lower respiratory tract by inhalation, the compounds of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more of the anti-cancer compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of the invention may be administered by means of nose drops or a liquid spray, such as by means of a plastic bottle atomizer or metered-dose inhaler. Examples of atomizers are the Mistometer (Wintrop) and Medihaler (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The dosage formulations provided by this invention may contain the therapeutic compounds of the invention, either alone or in combination with other therapeutically active ingredients, and pharmaceutically acceptable inert excipients. The term ‘pharmaceutically acceptable inert excipients’ includes at least one of diluents, binders, lubricants/glidants, coloring agents and release modifying polymers.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

The dosage form may include one or more diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

The dosage form may include one or more binders in an amount of up to about 60% w/w. Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscarmellose sodium, crospovidone, low substituted hydroxypropyl cellulose, and the like. The concentration may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration may vary from 0.1% to 15%, by weight, of the dosage form.

Release modifying polymers may be used to form extended release formulations containing the therapeutic compounds of the invention. The release modifying polymers may be either water-soluble polymers, or water insoluble polymers. Examples of water-soluble polymers include polyvinylpyrrolidone, hydroxy propylcellulose, hydroxypropyl methylcellulose, vinyl acetate copolymers, polyethylene oxide, polysaccharides (such as alginate, xanthan gum, etc.), methylcellulose and mixtures thereof. Examples of water-insoluble polymers include acrylates such as methacrylates, acrylic acid copolymers; cellulose derivatives such as ethylcellulose or cellulose acetate; polyethylene, and high molecular weight polyvinyl alcohols.

Another embodiment of the invention relates to the use of any of the compounds or compositions of the invention in the preparation of a medicament for the treatment of cancer.

Another embodiment of the invention relates to the use of any of the compounds or compositions of the invention in the preparation of a medicament for the inhibition of the growth or metastasis of a cancer in a mammal.

Another embodiment of the invention relates to the use of any of the compounds or compositions of the invention for use in the treatment of a cancer. Another embodiment of the invention relates to the use of any of the compounds or compositions of the invention for the inhibition of the growth or metastasis of a cancer in a mammal.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Drug Design Methodology.

Design of the competitive inhibitors of TopoIIα began by applying the 1.87Å TopoIIα crystal structure (Ponder, J.; Yoo, B. H.; Abraham, A. D.; Li, Q.; Ashley, A. K.; Amerin, C. L.; Zhou, Q.; Reid, B. G.; Reigan, P.; Hromas, R.; Nickoloff, J. A.; LaBarbera, D. V., Neoamphimedine Circumvents Metnase-Enhanced DNA Topoisomerase IIα Activity Through ATP-Competitive Inhibition. Marine Drugs 2011, 9:2397-2408) using the CHARMm forcefield, with residues corrected for physiological pH. The binding site was defined as whole residues within an 8Å radius subset encompassing the ATPase site. LigandFit and Flexible Docking protocols were used for the molecular docking of neoamphimedine (neo) and derivatives into the defined binding site of TopoIIα. Grid resolution was set to 0.5Å and electrostatic energy was included in the calculation of ligand internal energy. In order to avoid identical conformations, a root mean square deviation threshold of 1.5Å and a score threshold of 20 kcal/mol were used. The appropriate number of structural outputs up to fifty was specified and the identification of a docked conformation was followed by a minimization using the conjugate gradient method to a convergence of 0.001 kcal/mol to optimize ligand-protein interactions. Non-solvent binding, Generalized Born, and Poisson-Boltzmann methods were used to calculate binding energies, with a non-bonded list radius of 12Å. The top-ranked conformations for each docked complex were selected on the following: calculated binding energies, hydrogen bond, and pi interactions between the protein and ligand.

### Example 2

### The Synthesis of Two Neo Derivatives

The synthetic methodology to prepare neo has been shown previously (LaBarbera D V, Bugni T S, Ireland C M (2007), The Total Synthesis of Neoamphimedine. J Org Chem 72: 8501-8505). Derivatives of the core structure may be prepared using variations on these synthetic methods.

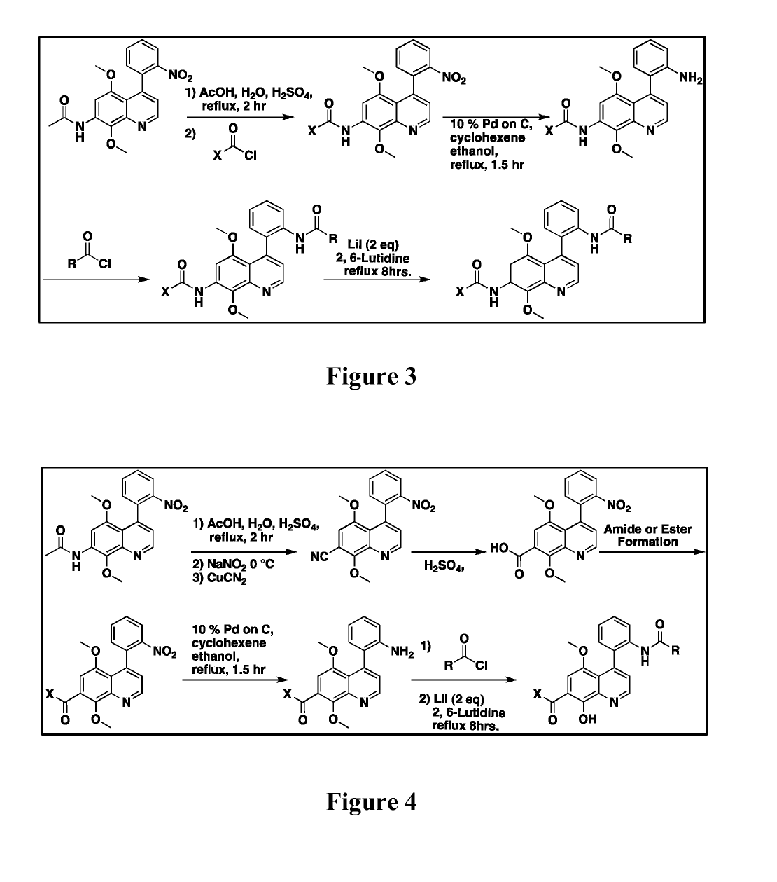

A first derivative, designated AA-67, is prepared using the synthetic route shown in FIG. 3. The acetamide starting material is hydrolyzed in aqueous acid under refluxing conditions for 2 hours. The resulting aniline is then functionalized (X) using the appropriate acid chloride or anhydride determined from molecular modeling. Subsequent catalytic reduction of the nitro group in refluxing cyclohexene and ethanol yields the amine, which can then be functionalized (R) with the appropriate acid chloride or anhydride.

A second derivative, designated AA-26, is synthesized beginning with the Sandmeyer reaction, prepared in one pot from the key neo intermediate (FIG. 4). The acetanilide is hydrolyzed as above. The corresponding aniline is then diazatized at 0° C. with sodium nitrite. The neutralized diazonium salt is added to a freshly prepared solution of potassium cuprous cyanide at 50° C. affording the nitrile in 58% yield. The nitrile is converted to an acid by first generating the primary amide in situ at 70° C. for 3 h with concentrated sulfuric acid followed by heating at 120° C. for 2 hours resulting in 80% yield. Amides may be prepared from the acid intermediate using peptide coupling with EDC. Once the acid is derivatized (X) the nitro is reduced and functionalized (R) using the method described for AA-67, and final selective demethylation of the 8-methoxy group to yield AA-26 and derivatives.

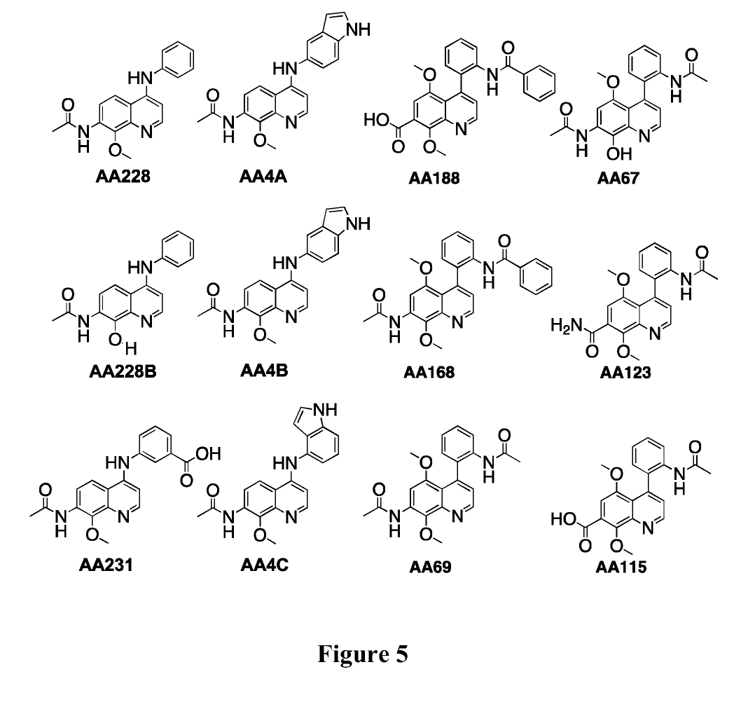

Once validated, the model was used to screen additional compounds in silico, and 12 of the derivatives identified were synthesized, and tested against TopoIIα. The structure of the 12 derivatives synthesized is shown in FIG. 5.

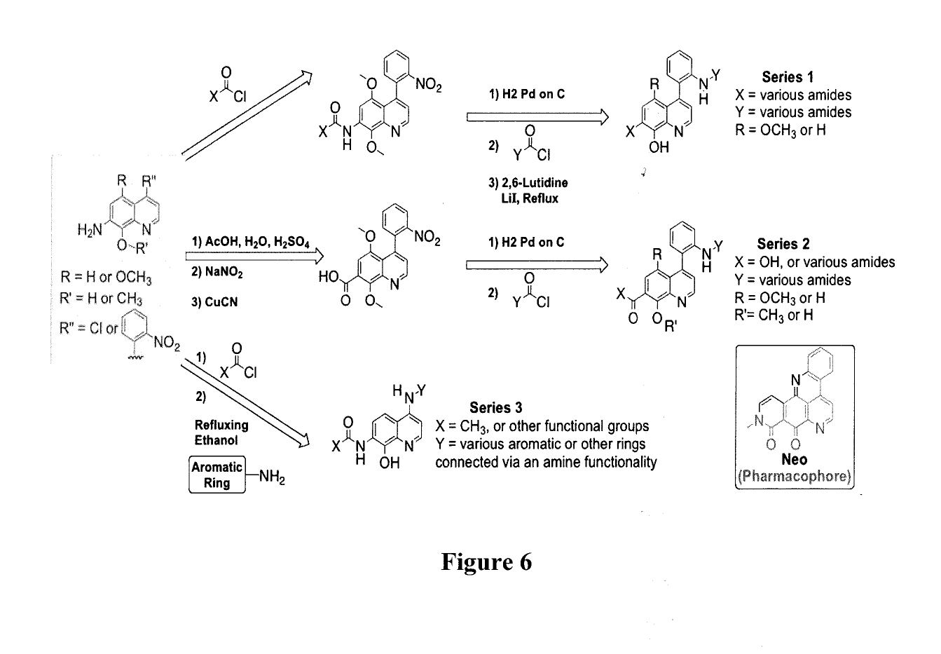

The inventor has previously published the synthetic methodology to prepare neo and derivatives (LaBarbera, D. V.; Bugni, T. S.; Ireland, C. M., The Total Synthesis of Neoamphimedine. J Org Chem 2007, 72, 8501-8505). Referring to FIG. 6, the precursor (shaded box) is functionalized (X) using the appropriate acid chloride/anhydride. Subsequent reduction of the nitro group in refluxing cyclohexene and ethanol will yield the amine that is functionalized (Y) with the appropriate acid chloride or anhydride, giving derivatives of series 1.

Derivatives of series 2 are obtained starting with the Sandmeyer reaction, prepared in one pot from the aniline precursor, which is diazatized at 0° C. with sodium nitrite. The neutralized diazonium salt is added to potassium cuprous cyanide at 50° C. affording the nitrile, which is hydrolyzed to the carboxylic acid in concentrated sulfuric acid. Once the acid is derivatized (X) via peptide coupling with EDC the nitro is reduced and functionalized (Y) using the method described for series 1.

Derivatives of series 3 are made from the precursor where R″ is Cl, which is substituted with various amines via nucleophilic aromatic substitution in refluxing ethanol (e.g. derivative AA4A).

### Example 3

### Testing Inhibitors of Topoisomerase

A. In Vitro ATP Hydrolysis Assay

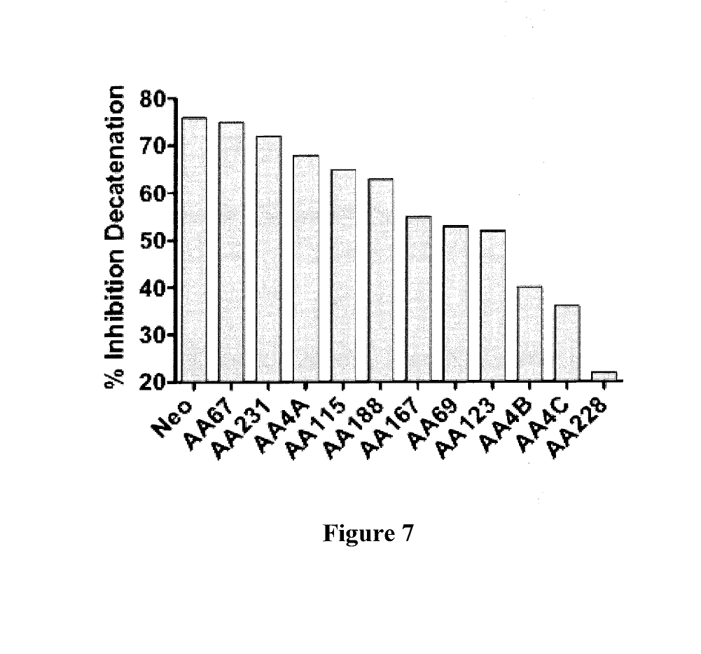

Efficacy of the ATP-competitive inhibition of TopoIIα inhibitors of the invention are tested in vitro using an ATP hydrolysis assay that quantifies inorganic phosphate through the formation of a high molar absorptivity complex with malachite green and molybdate, adapted for use in 384-well plates, with a total reaction volume of 15 μL. This assay was first utilized to determine the Kₘ of TopoIIα for its natural substrate, ATP, using ATP concentrations ranging from 62.5-1600 μM. To measure the apparent Kₘ, the same reactions were repeated in the presence of 2, 5, or 10 μM neo. Absorbance at 620 nm was measured using a microtiter plate reader. To determine the Kᵢ the same method was used with the following modifications: inhibitors (0.1-10 μM) in reaction buffer (without DNA and DTT) are added to the complete reaction buffer and preincubated for 10 min at 37° C. Reactions were initiated by the addition of 600 μM ATP and incubated for 30 min at 37° C. The IC₅₀ value was determined and the Kᵢ was calculated using the Cheng-Prusoff equation. Experiments were repeated in triplicate and GraphPad Prism software was used to perform non-linear fits (method of least squares) to the Michaelis-Menten kinetics (Kₘ and Vₘₐₓ) or the dose-response inhibition model (IC₅₀ values). In vitro testing results for the 12 derivatives shown in FIG. 5 are shown in FIG. 7. As shown there, all 12 compounds display activity, but AA67, AA231, AA4A, AA115, and AA188 are comparable to neo.

B. DNA Decatenation Assay

DNA decatenation assays are used as an additional measure to determine inhibitory activity against TopoIIα. TopoIIα is added to kinetoplast DNA (kDNA) containing small interlocked supercoiled circular DNA in reaction buffer (50 mM Tris-HCl, pH 8.0, 120 mM KCl, 10 mM MgCl₂, 0.5 mM DTT, 0.5 mM ATP) in the presence and absence of neo and derivatives using various concentrations. Reaction mixtures were incubated at 37° C. for 30 min. The reactions were then separated on a 1% agarose gel containing 0.5 μg/mL ethidium bromide with decatenated circular DNA, linear DNA and decatenated supercoiled DNA markers (TopoGen).

C. 3D-Spheroid Culture

A 3D-spheroid culture formulation was developed that allows for the culture of single uniform spheroids with variety of cell types in 96-well plates that can be used with a variety of epithelial-mesenchymal transition (EMT) biomarker reporters (see, U.S. Patent Publication No. 2011/0143960 A1; Li Q, Chen C, Kapadia A, Zhou Q, Harper M K, et al. (2011), 3D Models of epithelial-mesenchymal transition in breast cancer metastasis: high-throughput screening assay development, validation, and pilot screen. J Biomol Screen 16: 141-154). The 3D-spheroid based assays with neo (control) and derivatives were used to test their ability to revert EMT or induce cytotoxicity against human carcinomas resistant to clinically used TopoIIα drugs, including: MDA-MB-231, MCF7/AdrVP breast cancer, and SW480, Colo201, and DLD-1 (APC mutant) colon cancer cells. This 3D-model utilizes a luciferase reporter of vimentin, a key mesenchymal biomarker expressed in these cells. Vimentin expression is measured using a live spheroid luciferin substrate (caged luciferin) followed by a multiplex assay measuring spheroid viability. This dual assay allows assessment of whether neo and derivatives modulate EMT and the metastatic phenotype, as well as their effect on spheroid viability. Briefly, uniform single spheroids are cultured in 96-well flat-bottom plates coated with 50 μL of a 1.5% agarose (weight/volume) solution in serum-free RPMI-1640 (no phenol red) medium (freshly autoclaved at 121° C. for 15 min). During the coating process, the agarose/RPMI-1640 solution is maintained at ≧60° C. followed by cooling and setting at room temperature for 40 min. Then cells were plated at a density of 10,000 cells/well in 50 μL of RPMI-1640 (10% FBS, no phenol red) and centrifuged at 1000 rpm for 15 min to induce cell aggregation. The aggregated cells were then coated with 50 μL of a 10% solution of growth factor-reduced MATRIGEL™ (protein concentration: 7.1 mg/mL) in cell culture medium, resulting in a final volume of 100 μL with 5% MATRIGEL™. Spheroids were cultured for 4 days to reach an average diameter of 500 μm under standard tissue culture conditions (37° C., 5% CO2) without changing or adding medium.

VimPro-Fluc-MDA-MB-231 spheroids were cultured in 96-well optical bottom white plates and treated with neo and derivatives on day 5 using various concentrations. On day 8, after 72-h treatment, live spheroid vimentin expression is measured by adding 10 μL (final 12.5 μM) of DMNPE-caged luciferin to each well and incubating at 37° C., 5% CO₂ for 2 hours. Untreated day 5 spheroids were used as an additional negative control, and day 8 spheroids treated with 10% Triton X-100 at 37° C. for 2 hours were used as a control to induce spheroid death. Luminescence is detected using an ENVISION™ plate reader (Perkin Elmer).

Spheroid viability is assessed using the acid phosphatase (APH) colorometric (OD₄₀₅) assay adapted to be multiplexed with vimentin expression in VimPro-Fluc spheroids as follows: directly after measuring live VimPro-Fluc-MDA-MB-231 spheroid vimentin expression, cell viability was measured by adding 125 μL of APH assay buffer (0.1 M sodium acetate, 0.1% Triton X-100, supplemented with 10 mM p-nitrophenyl phosphate) followed by incubating for 90 min at 37° C. The optical density (OD₄₀₅) was measured directly after adding 10 μL of 1N NaOH to each well using an ENVISION™ plate reader.

Active compounds are then tested for their ability to inhibit spheroid invasion, as follows: Spheroid invasion is measured using 15 spheroids per sample group. The spheroids for each sample group are pooled, and centrifuged (10,000 rpm, 10 min at RT). Each sample group is then treated with 200 μL of spheroid dissociation solution (0.25% trypsin-EDTA) and incubated at 37° C. for 30 minutes. Single cell suspensions of each group in RPMI-1640 medium (0.1% BSA) were seeded at 35,000 cells/chamber (50 μL) into the top chambers of a 96-well invasion plate previously coated with 50 μL of MATRIGEL™ (40 μg/mL protein in RPMI-1640). Chemoattractant (10% FBS in RPMI, 150 μL) is added to each bottom chamber and incubated (37° C., 5% CO₂) for 24 h. Cells that did not invade are carefully removed from the top chamber using a cotton swab. Each side of the membrane insert was washed 2× by adding 200 μL PBS-CMF (Ca/Mg free) to the bottom chamber, replacing the insert and adding 50 μL PBS-CMF to the top chamber. Invading cells were fixed with 200 μL of 4% paraformaldahyde in the bottom chambers. The insert was washed 2× with PBS-CMF and were subsequently stained with 200 μL of DAPI (3 μg/mL) in PBS-CMF for 30 min RT and washed 4×, as described above, in 15 min intervals. Invading cells will be measured using an OPERETTA™ high content imaging system.

### Example 4

### In Vivo Breast Cancer Model.

Neo and synthesized derivatives are tested side by side. Compounds are administered intraperitoneally (IP) at a dose of 50 mg/kg once a day, 5 times per week consecutively, for 22 days. This dose and schedule were selected based on previously reported in vivo studies with neo using tumor xenografts (Marshall K M, Matumoto S S, Holden J A, Concepcion G P, Tasdemir D, et al. (2003) The anti-neoplastic and novel topoisomerase II-mediated cytotoxicity of neoamphimedine, a marine pyridoacridine. Biochem Pharmacol 66: 447-458). Twenty animals are inoculated for each experimental group. An 80-90% take rate is expected and 16-18 animals are divided into control and treatment groups. Metastasis is measured in vivo using a stably engineered VimPro-Fluc-MDA-MB-231-GFP cell line that expresses a luciferase reporter driven by the vimentin promoter and a green fluorescent protein (GFP) selection marker. Vimentin is a biomarker of the mesenchymal phenotype and vimentin expression correlates with the metastatic potential of MDA-MB-231 cells and thus makes a good reporter for metastasis. An intracardiac injection method is used to deliver breast cancer cells into the circulatory system. Anesthetized, immune-compromised mice are inoculated into the left ventricle with 10⁵ cells suspended in 100 μL of sterile PBS with a 27.5 gauge needle. This results in metastatic lesions to common sites of breast cancer metastasis (brain, bone, lung, etc). To visualize and quantitate the size of metastatic lesions, anesthetized mice are injected (IP) with 75 mg/kg of D-luciferin/PBS and bioluminescent images immediately acquired using the IVIS imaging system.

These experiments determine if neo and derivatives have therapeutic value in treating existing metastases. For these experiments, animals are injected with MDA-MB-231 cancer cells as described above and metastases are established prior to treating with neo and derivatives. Seven days post injection, animals are monitored for implantation of tumor cells at distal sites. These animals are then randomized into control and treatment groups. Animals then receive placebo, or neo, or derivatives. During treatment time, animals will undergo IVIS imaging twice weekly to confirm that neo and derivatives can reduce existing breast cancer metastases.

### Example 5

### In Vivo Colon Cancer Model

The mutant APC^{min/+} mouse model is used to mimic Human familial adenomatous polyposis (FAP) and spontaneously form intestinal and colon polyps that may become invasive. Min mice have a germ-line nonsense mutation at codon 850 of adenomatous polyposis coli (Apc) gene and spontaneously develop multiple polyps in the small and large intestines at the age of 10-12 weeks. The Min mouse is used in conjunction with a well-established chemical-induced colon cancer model in rodents, which is based on the administration of colon carcinogen azoxymethane (AOM) and has been shown to cause efficient induction of colon tumors in wild-type mice. More importantly, treatment of Min mice with AOM represents an experimental model, which more closely resembles human colon cancers in terms of distribution and histological features of the tumors. The effect of neo (control) and its potent derivatives is tested for ability to prevent colon carcinogenesis or treat existing tumors displaying the metastatic phenotype characterized by histology, as follows: Neo and derivatives are tested side by side. Compounds are administered intraperitoneally (IP) at a dose of 50 mg/kg once a day, 5 times per week consecutively, for 22 days. This dose and schedule was selected based on previously reported in vivo studies with neo using tumor xenografts. Seventeen experimental groups (2 genotypes by 8 treatments) are established, each of which comprise twenty male mice of 6-8 weeks old. Two genotype groups include wild type and Apc^{min/+} mutant mice. The MIN mice are available from the Jackson's Lab and are in C57BL/6J background. Eight treatment groups include control (saline), AOM alone, neo or derivatives alone, and AOM plus neo or derivatives (two treatment windows for each tested compound). Colon tumors are induced by AOM treatment, which includes six consecutive weekly injections of AOM (10 mg/kg, IP). Neo and its derivatives are administered in two separate windows, starting either simultaneously with AOM treatment (window I, prevention) or a week after the last AOM administration (window II, treatment). Control groups receive injections of the same amount of saline during the treatment period. All animals are killed 10 weeks after the last AOM administration for tumor evaluation.

Mice are sacrificed and their large bowels flushed with saline, excised and measured for length. The large bowels will are then cut open longitudinally along the main axis, washed with saline and grossly inspected for tumor incidence, distribution and size. Finally, the excised large bowels are processed for histological and biochemical examination.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art

1. A compound having the structure of Formula I: 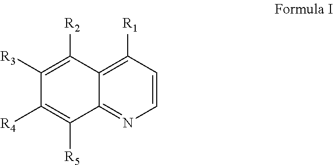
and pharmaceutically acceptable salts, solvates, tautomers, racemates, pure enantiomers, diastereoisomers or N-oxides thereof,
wherein:
R₁ is: H, —NO₂, —NHC(O)R₁₀, —NH₂, —NHR₈, or —NR₈R₉;
or R₁ is: phenyl or substituted phenyl, wherein the substituents are independently selected from amides, —NCOCH₃, —NCOR₁₃, —NCOR₁₃, and —C₁₋₆ alkyl;
R₂ is: H, —OR₁₄ or —NR₁₄, wherein R₁₄ is halogen, C₁₋₆ alkyl, C₃₋₆ cycloalkyl, cyano, or optionally substituted phenyl, wherein the optional substituents are independently selected from halogens, hydroxyl, C₁₋₆ alkoxy;
R₃ is: H, halogen, C₁₋₆ alkyl, C₃₋₆ cycloalkyl, or cyano;
R₄ is: H, sulfonamide, phosphamide, sulfonate ester, phosphonate ester, P-amido-imido-diphosphoric acid, imido-disulfamide, —NHR₈, —NHR₈R₉; —NH—SO₂—R₁₁, NH—PO₂—R₁₁, —COR₁₂, or —NHCOR₁₂;
or R₃ and R₄ together form a C₅₋₁₀ membered optionally substituted cycloalkyl, aryl or heteroaryl ring with one or more heteroatoms independently selected from nitrogen, oxygen and sulfur, and wherein the optional substituents are independently selected from halogens, hydroxyl, C₁₋₃ alkoxy, cyano, C₁₋₆ alkyl or C₃₋₆ cycloalkyl, and aryl;
R₅ is: H, —OR₁₅ or —NR₁₅, wherein R₁₅ is halogen, C₁₋₆ alkyl, C₃₋₆ cycloalkyl, or cyano;
R₈ and R₉ are independently selected from H, or optionally substituted C₁₋₆ alkyl, C₄₋₁₀ aryl, phenyl or indole, wherein the optional substituents are independently selected from halogens, hydroxy, C₁₋₃ alkoxy, phenyl or substituted phenyl, wherein the substituents are individually selected from amides, —NCOCH₃, —NCOR₁₃, and —C₁₋₆ alkyl;
R₁₀ is: H or optionally substituted C₁₋₆, phenyl, benzyl, and naphthyl, imidazole, pyridyl, pyrimidine, pyrolidine, quinoxaline, and quinoline ring systems wherein the optional substituents are independently selected from amino, halogens, hydroxyl, C₁₋₆ alkoxy;
R₁₁ and R₁₂ are independently selected from H, hydroxyl, optionally substituted C₁₋₆ alkyl, phenyl, benzyl, and naphthyl, imidazole, pyridyl, pyrimidine, pyrolidine, quinoxaline, and quinoline, wherein the optional substituents are independently selected from amino, halogens, hydroxyl, and C₁₋₆ alkoxy; and,
R₁₃ is: C₁₋₆ alkyl, C₄₋₆ aryl.

2. A compound of claim 1, wherein
R₁ is —N—C(O)CH₃,
R₂ is —OCH₃,
R₃ is —H,
R₄ is —COOH, —C(O)NH₂, —NHC(O)CH₃ and
R₅ is —OH.

3. A compound of claim 1, wherein
R₁ is —CH₃,
R₂ is —OCH₃,
R₃ is —H,
R₄ is —NHCOCH₃, and
R₅ is —OH.

4. A compound of claim 1, having the structure: 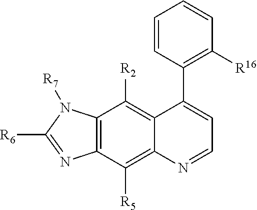
or pharmaceutically acceptable salts, solvates, tautomers, racemates, pure enantiomers, diastereoisomers or N-oxides thereof,
wherein:
R₁₆ is: amide, —NCOCH₃, —NCOR₁₃, —NCOR₁₃, and —C₁₋₆ alkyl;
R₂ is: H, —OR₁₄ or —NR₁₄, wherein R₁₄ is halogen, C₁₋₆ alkyl, C₃₋₆ cycloalkyl, cyano, or optionally substituted phenyl, wherein the optional substituents are independently selected from halogens, hydroxyl, C₁₋₆ alkoxy;
R₅ is: H, —OR₁₅ or —NR₁₅, wherein R₁₅ is halogen, C₁₋₆ alkyl, C₃₋₆ cycloalkyl, or cyano;
R₆ is: sulfonamide, phosphamide, sulfonate ester, phosphonate ester, P-amidoimidodiphosphoric acid, imidodisulfamide, —N—SO₂—R₁₁, —N—PO₂—R₁₁—C(O)R₁₁, or —NHC(O)—R₁₂;
or R₆ is C₁₋₆ alkyl optionally substituted with sulfonamide, phosphamide, sulfonate ester, phosphonate ester, P-amidoimidodiphosphoric acid, imidodisulfamide, —N—SO₂—R₁₁, —N—PO₂—R₁₁ —C(O)R₁₁, or —NHC(O)—R₁₂; and,
R₇ is: C₁₋₆ alkyl, optionally substituted with halogens, hydroxy, or C₁₋₃ alkoxy.

5. A pharmaceutical composition comprising at least one compound of claim 1 and at least one pharmaceutically acceptable carrier.

6. A method of treating or ameliorating a cancer, or preventing a cancer metastases, comprising administering to a mammal in need of such treatment, a therapeutically effective amount of a compound that inhibits ATP binding to the N-terminal ATPase binding site of TopoIIα.

7. A method of treating a cancer in a mammal, comprising administering to a mammal having a cancer a compound of claim 1.

8. (canceled)

9. The method of claim 7, wherein the compound is administered to the mammal in a pharmaceutical composition.

10. The method of claim 9, wherein the pharmaceutical composition is a mono-phasic pharmaceutical composition suitable for parenteral or oral administration consisting essentially of a therapeutically-effective amount of the compound, and a pharmaceutically acceptable carrier.

11. The method of claim 7, wherein the cancer is a cancer selected from the group consisting of breast, prostate, ovarian, small cell lung, cervical, neuroblastoma, endometrial, melanoma, renal and peritoneal cancers.

12. The method of claim 7, wherein the compound is administered in conjunction with surgery, chemotherapy, radiation, immunotherapy, or combinations thereof.

13. A pharmaceutical composition comprising a therapeutically-effective amount of the compound of claim 1, and a pharmaceutically acceptable carrier.

14-16. (canceled)
